(19) United States
(12) Patent Application Publication
(10) Pub. No.: US 2023/0302177 A1
(43) Pub. Date: Sep. 28, 2023

(54) MEDICAL END-SITE DISINFECTING DEVICE WITH CAPILLARY ACTION

(71) Applicant: 1WorldVista Medical, Inc., Rio Verde, AZ (US)

(72) Inventor: Michael J. Ferlic, Roseville, MN (US)

(73) Assignee: 1WorldVista Medical, Inc., Rio Verde, AZ (US)

(21) Appl. No.: 17/889,469

(22) Filed: Aug. 17, 2022

Related U.S. Application Data
(60) Provisional application No. 63/323,336, filed on Mar. 24, 2022.

(51) Int. Cl. A61L 2/18

(52) U.S. Cl. CPC A61L 2/18; A61L 2202/24

(57) ABSTRACT

A medical end-site disinfecting device including an interface device and a volume of liquid disinfectant. The interface device is configured to interface with a medical end-site, and includes at least one of a housing, an element, and a core. At least one component of the interface device defines at least one capillary channel. The volume of liquid disinfectant is retained along the capillary channel. The capillary channel is configured to generate a capillary effect to transport the liquid disinfectant along the interface device. In some embodiments, the capillary channel is configured to retain at least a portion of the volume of liquid disinfectant along a length of the capillary channel, for example to apply the retained volume of the liquid disinfectant device onto the medical end-site during use.

### CROSS-REFERENCE TO RELATED APPLICATIONS

This application claims the benefit of the filing date of U.S. Provisional Application No. 63/323,336, filed Mar. 24, 2022, the entire teachings of which are incorporated herein by reference.

### BACKGROUND

The present disclosure relates to devices and methods for sterilizing the medical connection sites of luer connections, luer compatible components, catheter hubs, and other medical connections and access ports. More particularly, it relates to devices and methods for readily distributing a contained liquid disinfection solution onto a component being sterilized.

Mating luer connections, needleless connectors, and needle access ports serve as a conduit for administering medication to a patient by the joining of their mutual complimentary components. Prior to connecting two luer compatible components together, it is important to sterilize the connection end-sites. Typically, the connection end-sites are sterilized by wiping each site with an antiseptic wipe. Contacting and cleaning intricate details on an end-site such as cracks, crevices or grooves and where microscopic bacterium can reside on an end-site, and particularly, where an end-site has been assembled with multiple components having microscopic surfaces that can harbor bacterium (e.g. needle-less connector having assembled components such as a housing, seals, valve or septum) requires an awareness to effectively sterilize and thoroughly kill those pathogens that would otherwise make an already sick patient worse. The wiping and sterilizing of the connection end-sites must be done for a specified amount of time and accuracy to achieve a “kill of microbes” prior to the luer compatible components being connected together to reduce the risk of infection to the patient. This is also true for needle access ports or other connections. Without this simple precautionary step of sterilizing the working end-sites, patients are at a greater risk of contracting an infection.

The current method for sterilizing a connection end-site, catheter hub, needle access port, or needleless connector employs an antiseptic towelette that comes in a small foil packet and is commonly used throughout hospitals, clinics, and home healthcare. The foil packet in which the antiseptic towelette comes in must be torn open and the towelette lifted out with gloved hands. The towelette is a small folded sheet of fibrous, non-woven material that contains isopropyl alcohol. The clinician cannot adequately use the towelette to wipe the various complex surfaces, edges, threads, lumen, septum of a working end-site due to the towelette's small size and flimsy characteristics. Thus, that which should be a routine precautionary step to maintain sterility is unfortunately either ignored or not adequately performed to prevent patient infection.

### SUMMARY

The inventor of the present disclosure has recognized a need to address one or more of the above-mentioned problems.

Some aspects of the present disclosure are directed to a medical end-site disinfecting device including an interface device and a volume of liquid disinfectant. The interface device is configured to interface with a medical end-site, and includes at least one of a housing, an element, and a core. At least one component of the interface device defines at least one capillary channel. The volume of liquid disinfectant is retained along the capillary channel. The capillary channel is configured to generate a capillary effect to transport the liquid disinfectant along the interface device. In some embodiments, the capillary channel is configured to retain at least a portion of the volume of liquid disinfectant along a length of the capillary channel, for example to apply the retained volume of the liquid disinfectant device onto the medical end-site during use. In some embodiments, the interface device include the core disposed within a chamber of the element. With these and related embodiments, the core is formed of a non-absorbent closed-cell foam and the element is formed of a non-absorbent elastic material.

### BRIEF DESCRIPTION OF THE DRAWINGS

FIG. 1 is an exploded perspective view of a medical end-site disinfecting device in accordance with principles of the present disclosure and including an interface assembly;

FIG. 2 is an enlarged cross-sectional view of an element useful with the interface assembly of FIG. 1;

FIG. 3A is an enlarged perspective view of a core useful with interface assembly of FIG. 1;

FIG. 3B is a top view of the core of FIG. 3A;

FIG. 3C is a cross-sectional view of the core of FIG. 3B, taken along the line 3C-3C;

FIG. 4 is a perspective view of a core in accordance with principles of the present disclosure and useful with the interface assembly of FIG. 1;

FIG. 5 is a perspective view of a core in accordance with principles of the present disclosure and useful with the interface assembly of FIG. 1;

FIG. 6 is a perspective view of a core in accordance with principles of the present disclosure and useful with the interface assembly of FIG. 1, and illustrating a capillary effect;

FIG. 7 is a perspective view of a core in accordance with principles of the present disclosure and useful with the interface assembly of FIG. 1, and illustrating a capillary effect;

FIG. 8 is a perspective view of a core in accordance with principles of the present disclosure and useful with the interface assembly of FIG. 1, and illustrating a capillary effect;

FIG. 9 is a perspective view of a core in accordance with principles of the present disclosure and useful with the interface assembly of FIG. 1, and illustrating a capillary effect;

FIG. 10 is a perspective view of a core in accordance with principles of the present disclosure and useful with the interface assembly of FIG. 1, and illustrating a capillary effect;

FIG. 11 is a perspective view of a core in accordance with principles of the present disclosure and useful with the interface assembly of FIG. 1, and illustrating a capillary effect;

FIG. 12 is a perspective view of a core in accordance with principles of the present disclosure and useful with the interface assembly of FIG. 1, and illustrating a capillary effect;

FIG. 13A is a simplified representation of a capillary channel formed in a core component of the interface assembly of FIG. 1, viewed from an end of the core;

FIG. 13B is a simplified representation of a capillary channel formed in a core component of the interface assembly of FIG. 1, viewed from an end of the core;

FIG. 13C is a simplified representation of a capillary channel formed in a core component of the interface assembly of FIG. 1, viewed from an end of the core;

FIGS. 14A and 14B are cross-sectional views illustrating final construction of the interface assembly of FIG. 1;

FIG. 15 is a cross-sectional view of the disinfecting device of FIG. 1 upon final assembly;

FIG. 16 is a cross-sectional view of an element useful with the interface assemblies and disinfecting devices of the present disclosure, and forming at least one capillary channel;

FIG. 17 is a cross-sectional view of an element useful with the interface assemblies and disinfecting devices of the present disclosure, and forming at least one capillary channel;

FIG. 18 is a perspective, cross-sectional view of a housing useful with the interface assemblies and disinfecting devices of the present disclosure, and forming at least one capillary channel;

FIG. 19 is a cross-sectional view of another interface assembly in accordance with principles of the present disclosure;

FIG. 20 is a cross-sectional view of a housing useful with the interface assemblies and disinfecting devices of the present disclosure, and forming at least one capillary channel; and

FIGS. 21A-21K are block diagrams of interface assemblies in accordance with principles of the present disclosure and useful with a medical end-site disinfecting device.

### DETAILED DESCRIPTION

All medical luers and all medical device ends need to be sterilized prior to use. The term “luer” is well known in the medical field and in the art and is used here (luer hub, male luer, female luer, slip luer) to mean mating structures, with or without threads, that allows two mating luer devices, or luer compatible components, to be joined for fluid communication. The term “site,” “end-site” or “site end” is used interchangeably and is used here to be understood to mean any and all working ends and/or sites including, but not limited to, a luer, luer hub (e.g. catheter hub), luer compatible component, needle access port, needleless connector, or septum. According to various embodiments, aspects of the present disclosure provide a devices or tool for effectively sterilizing and wiping debris from all surfaces of a working end including, but not limited to, threads, sides, edges, inner lumens, septums, and needle access ports.

In some embodiments, the medical end-site disinfecting devices of the present disclosure generally include an interface assembly containing a supply or volume of a liquid disinfectant agent or anti-pathogenic agent (e.g., isopropyl alcohol). The interface assembly can assume various forms, and can consist of one, two or more components. At least one of the interface assembly components (including embodiments in which the interface assembly consists of a single component) forms or defines at least one capillary channel configured to facilitate mass flow or transport of the liquid disinfectant agent along a surface of the housing assembly component. The mass flow effected by the capillary channel(s) can also be referred to as capillary action, capillary effect, or wicking, and distributes the liquid disinfectant agent along surface(s) of the interface assembly component for ensuring contact between the liquid disinfectant agent and the medical end-site to which the device is applied. As used herein, including the claims, the phrase “capillary channel” means an elongated void of consistent, increasing, decreasing or fluctuating width in a non-absorbing material surface along which a liquid, for example a liquid disinfectant such as isopropyl alcohol, can flow without the assistance of, or even opposition to, external forces such as gravity by means of capillary action (e.g., the liquid wicks due to fine, narrow capillary channel(s) facilitating molecular surface tension and the ensuing capillary action sufficient to retain and transport (migrate) the liquid along the surface of the body forming the channel). The elongated void may be any of a cut, slit, score, sipe, groove, indent, impression, notch, channel, incision, interstice, crevice, cranny, fissure, depression, etc., integrally molded into, formed during molding or created post-formation by cutting, slitting, slicing, scoring, plasma etching, etc.

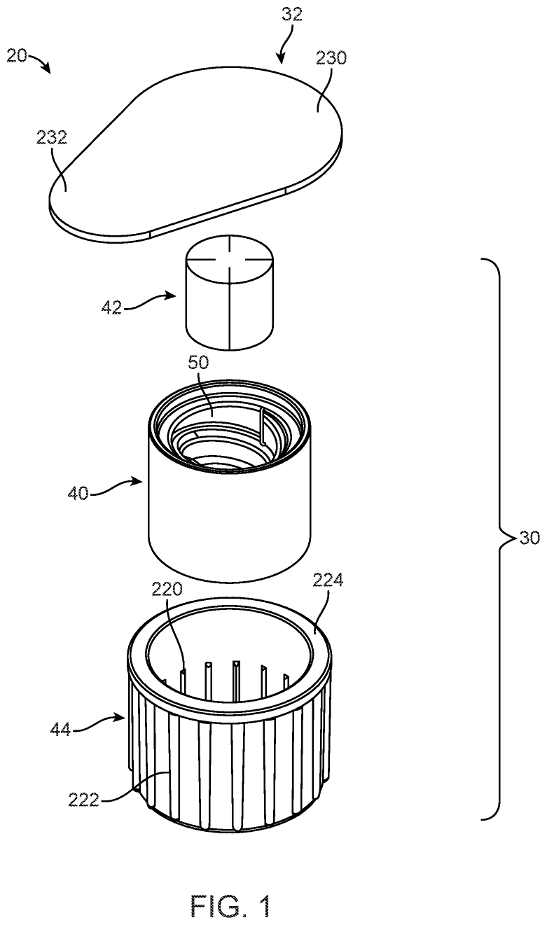

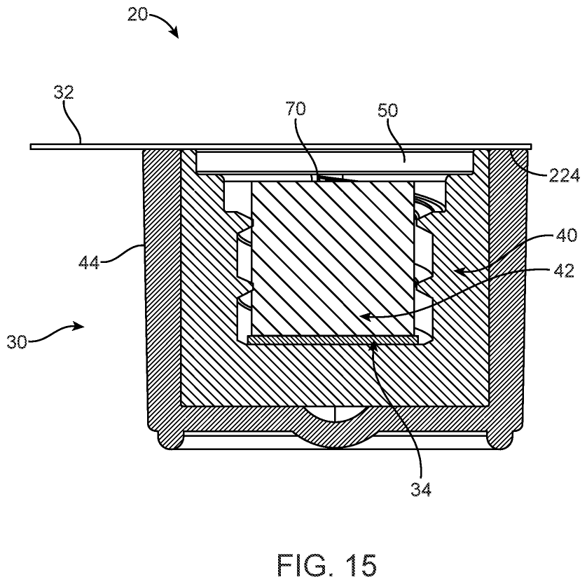

With the above in mind, one embodiment of a medical end-site disinfecting device 20 in accordance with principles of the present disclosure is shown in FIG. 1. The device 20 includes an interface assembly 30, a lid 32 and a volume of liquid disinfectant 34 (hidden in FIG. 1, but shown, for example in FIG. 15). Details on the various components are provided below. In general terms, the interface assembly 30 is generally configured to intimately interface with a medical end-site, and contains a volume of the liquid disinfectant 34 (e.g., isopropyl alcohol, povidone iodine, chlorhexidine gluconate, and other useful anti-pathogenic agents known to those of skill in the art). At least one component of the interface assembly 30 forms at least one capillary channel along which the liquid disinfectant 34 is caused to be transported via capillary action. The lid 32 is removably secured to the interface assembly 30 prior to use to maintain an integrity of the contained liquid disinfectant 34. To clean and disinfect a medical end-site, the lid 32 is removed from the interface assembly 30. A user directs the interface assembly 30 into contact with the medical end-site, manipulating the interface assembly 30 relative to the medical end-site in a manner to effect cleaning thereof. As the interface assembly 30 is manipulated to effect cleaning, the liquid disinfectant 34 is transferred or applied onto the medical end-site to disinfect contacted surface(s) of the medical end-site. In this regard, the capillary channel(s) better ensure that the liquid disinfectant 34 is present at or along regions of the interface assembly 30 likely to be in contact with medical end-site during use.

The interface assembly 30 can assume various forms, and in some embodiments includes an element or cap 40 (as used throughout this disclosure and claims, the terms “element” and “cap” are interchangeable), a core 42, and a housing 44. In general terms, the element 40 forms a chamber 50 sized to contain the core 42 and the volume of liquid disinfectant 34 (hidden in FIG. 1), and forms or provides one or more features configured to facilitate interface with a medical end-site. The core 42 is configured to provide enhanced cleaning attributes during use of the device 20 in cleaning/disinfecting a medical end-site. Finally, the housing 44 is sized and shaped to receive the element 40 (and thus the core 42), and can provide one or more features that facilitate or promote user handling.

The element 40 can assume various forms, and in some embodiments is formed (e.g., molded) of a non-absorbent (at least with respect to a composition of the liquid disinfectant) resilient material, such as an elastomeric material (e.g., thermoplastic vulcanized rubber such as a thermoplastic vulcanizate (TPV) or other thermoplastic elastomer (TPE)) or the like. In some embodiments, the element 40 is formed of a non-absorbent resilient material such as a medical grade, high performance TPE available from Celanese Corp. of Dallas, Tex. under the trade designation Santoprene™ (a fully dynamically vulcanized ethylene propylene diene monomer (EPDM) rubber in a thermoplastic matrix of polypropylene (PP)). The element 40 forms or defines the chamber 50 along with one or more features or shapes adapted to fit to the various male, female (inner lumen), slip luer, septum, port, or threaded configurations of a medical end-site to be sterilized, and wipe debris from the site while using a wiping and twisting motion. By optionally forming the element 40 from a resilient material, the element 40 can more readily conform to (e.g., form fit) the contours, mating features, etc., of the particular medical end-site being cleaned/sterilized.

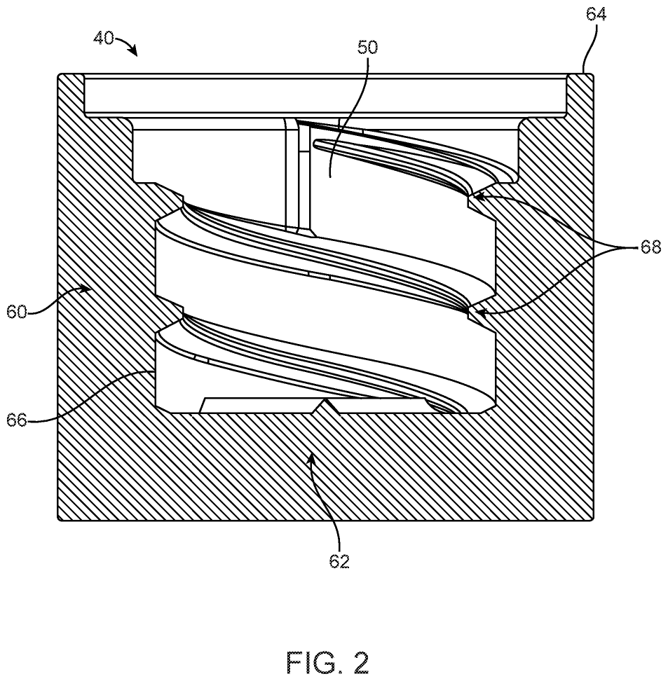

For example, and with additional reference to FIG. 2, the element 40 can include or define a side wall 60 extending between a base 62 and a leading end 64. The chamber 50 is circumscribed or defined by the side wall 60 and the base 62, and is open at the leading end 64. With this in mind, an inner surface 66 of the side wall 60 can form or define one more features intended to provide an enhanced interface with a medical end-site. For example, in some embodiments, a plurality of raised structures 68 are formed on or by the inner surface 66. The raised structures 68 project into the chamber 50, and are configured to engage the threads, sides, and/or edges on the working end-site. In some embodiments, the raised structures 68 can include threads, ridges, flanges, steps, nubs, tines, bumps, etc., or combinations thereof. The inner surface 66 can optionally define a tapering diameter (either stepped change in diameter or a more uniformly tapering diameter) in extension from the leading end 64 to the base 62. Regardless, the raised structures 68 located on the inner surface 66 can facilitate the effective cleaning (and sterilization) of surfaces of the medical end-site by directly contacting and conforming to surfaces of interest. In other embodiments, the inner surface 66 can have other constructions that may or may not include raised structures.

Returning to FIG. 1, in some embodiments the core 42 can be a solid body formed of non-absorbent (at least with respect to the liquid disinfectant formulation), closed-cell foam material. In some embodiments, the closed-cell foam material of the core 42 is non-porous, for example a non-porous, low density polyethylene closed-cell foam. Other, similar non-absorbent materials are also acceptable. In more general terms, the core 42 is non-absorbent and at least somewhat resilient or compressible, and provides a face configured to effect a more robust or aggressive cleaning of medical end-site surfaces (as compared to a material of the element 40) when in contact with and moved relative to the medical end-site surface.

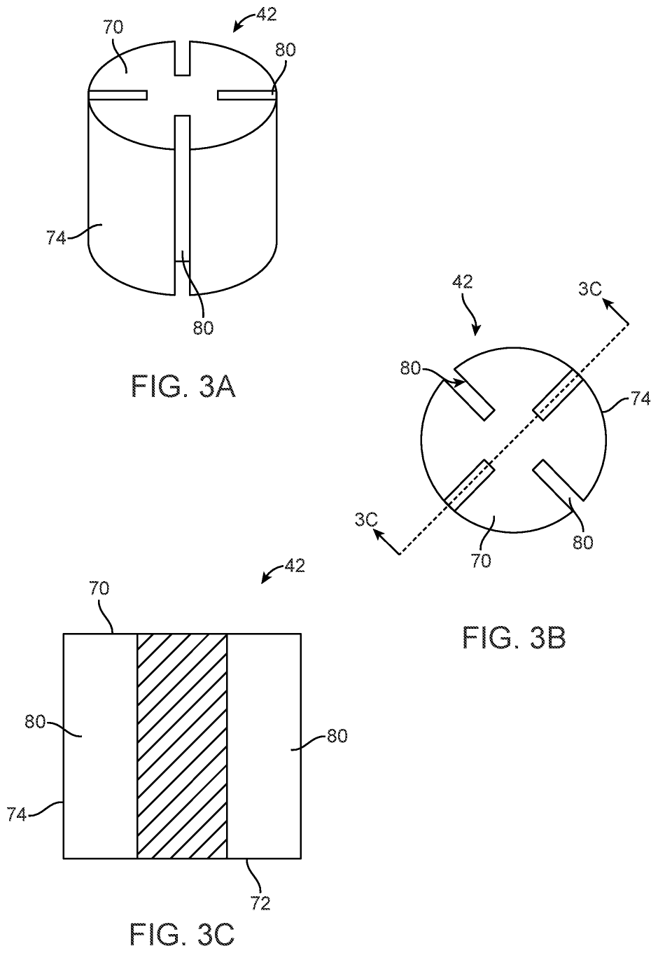

The core 42 can have the columnar, cylindrical shape shown, and is sized to nest within the chamber 50. With additional reference to FIGS. 3A-3C, an exterior of the core 42 can define opposing, first and second end faces 70, 72, and a side face 74 extending between the end faces 70, 72. In addition, one or more capillary channels 80 are formed in the core 42. With the non-limiting example of FIGS. 3A-3C, four of the capillary channels 80 are provided, each extending between, and open relative to, the end faces 70, 72 and the side face 74. Any other number, either greater or lesser, is equally acceptable. Where two (or more) of the capillary channels 80 are provided, the capillary channels 80 may or may not be identical in size and shape, and may or may not be equidistantly spaced from one another relative to a circumference of the core 42. With embodiments in which two (or more) of the capillary channels 80 are provided, the capillary channels 80 can be discrete from one another and do not intersect. In other embodiments, two or more of the capillary channels 80 can intersect other otherwise be open to one another. Regardless, the capillary channel(s) 80 are formed along the length of the core 42 and are configured to distribute the liquid disinfectant (not shown) along an outward-facing exterior surface of the core (e.g., one or both of the end faces 70, 72 and/or the side face 74).

The capillary channels of the present disclosure, for example the capillary channel(s) 80, can be formed or defined into the core 42 in various manners. For example, the capillary channel(s) 80 can be formed or defined as a cut, a groove, a sipe, split, a slit, a scoring, an indent, a notch, a channel, incision, fissure or another other format that produces a capillary effect and a transportation of liquid.

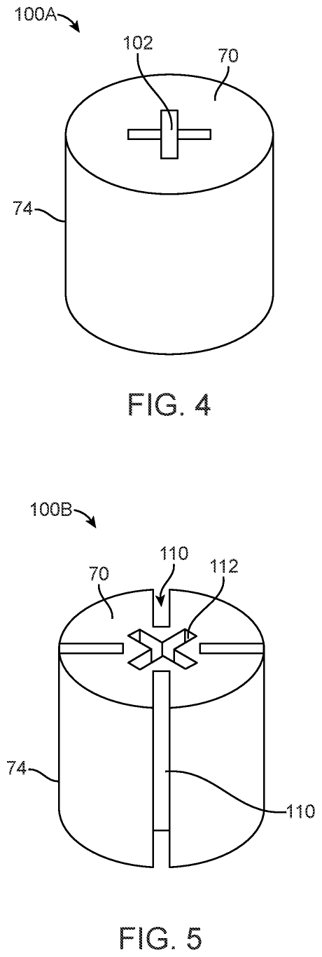

The format and arrangement of the capillary channels 80 of FIGS. 3A-3C is but one acceptable configuration in accordance with principles of the present disclosure. For example, another core 100A useful with the disinfecting devices of the present disclosure (e.g., in place of the core 42 of FIG. 1) is shown in FIG. 4. The core 100A can be akin to the core 42 in terms of material and shape (e.g., defines the opposing exterior end faces 70, 72 and the exterior side face 74 as described above). In addition, the core 100 forms or defines a capillary channel 102 generally in the form of a cross or “+” sign. The capillary channel 102 extends between, and is open relative to, the opposing end faces 70, 72, but is not open to the side face 74 (it being understood that the second end face 72 is hidden in FIG. 4). That is to say, in some embodiments, the capillary channel(s) of the present disclosure, as utilized with the core component, can be internal to a thickness of the core body. While capillary channel 102 is shown as having a cross-like shape, other shapes, regular or irregular, are also acceptable. Further, more than one of the capillary channels 102 can be provided.

Another core 100B useful with the disinfecting devices of the present disclosure (e.g., in place of the core 42 of FIG. 1) is shown in FIG. 5. The core 100B can be akin to the core 42 in terms of material and shape (e.g., defines the opposing exterior end faces 70, 72 and the exterior side face 74 as described above). In addition, the core 100B forms or defines one or more exterior capillary channels 110 and at least one interior capillary channel 112. The exterior capillary channel(s) 110 can be akin to the capillary channels 80 (FIG. 1) described above, open to the opposing exterior end faces 70, 72 (it being understood that the second end face 72 is hidden in FIG. 5), and to the exterior side face 74. The interior capillary channel 112 can be akin to the capillary channel 102 (FIG. 4) described above, and can generally be in the form of a cross or “+” sign extending between, and is open relative to, the opposing end faces 70, 72 (but is not open to the side face 74). While the interior capillary channel 112 is shown as having a cross-like shape, other shapes, regular or irregular, are also acceptable. Further, more than one of the interior capillary channels 112 can be provided.

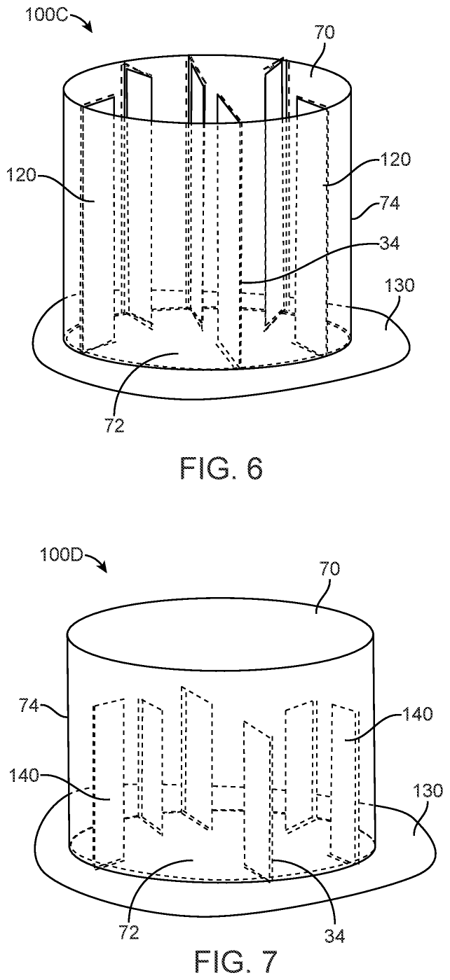

Another core 100C useful with the disinfecting devices of the present disclosure (e.g., in place of the core 42 of FIG. 1) is shown in FIG. 6. The core 100C is depicted as being transparent in the view of FIG. 6 to better illustrate the various features described below; it will be understood, however, that the material of the core 100C (e.g., a non-absorbent, closed-cell foam) need not be transparent in actual practice. The core 100C can be akin to the core 42 in terms of material and shape (e.g., defines the opposing exterior end faces 70, 72 and the exterior side face 74 as described above). In addition, the core 100C forms or defines one or more capillary channels 120. The capillary channel(s) 120 can be akin to the capillary channels 80 (FIG. 1) described above, open to the opposing exterior end faces 70, 72, and to the exterior side face 74. While six, equidistantly-spaced capillary channels 120 are shown, any other number, and any other spacing format or pattern, is equally acceptable. As a point of reference, FIG. 6 also depicts a capillary action. In particular, in the view of FIG. 6, the second end face 72 of the core 100C has been placed into a volume 130 of the liquid disinfectant 34. Dashed lines reflect that the liquid disinfectant 34 has transported along capillary channels 120 due to the capillary effect, and is distributed or exists at the first exterior end face 70 and the exterior side face 74.

Another core 100D useful with the disinfecting devices of the present disclosure (e.g., in place of the core 42 of FIG. 1) is shown in FIG. 7. The core 100D is depicted as being transparent in the view of FIG. 7 to better illustrate the various features described below; it will be understood, however, that the material of the core 100D (e.g., a non-absorbent, closed-cell foam) need not be transparent in actual practice. The core 100D can be akin to the core 42 in terms of material and shape (e.g., defines the opposing exterior end faces 70, 72 and the exterior side face 74 as described above). In addition, the core 100D forms or defines one or more capillary channels 140. The capillary channel(s) 140 can be generally akin to the capillary channels 120 (FIG. 6) described above, and are open to the second exterior end face 72 and to the exterior side face 74. With the embodiment of FIG. 7, however, one or more or all of the capillary channels 140 do not extend to, or are not open at, the first exterior end face 70. With these and related embodiments, the capillary channels 140 can be described or referred to as transecting the core 100D. While six, equidistantly-spaced capillary channels 140 are shown, any other number, and any other spacing format or pattern, is equally acceptable. As a point of reference, FIG. 7 also depicts the capillary action as described above, with the second end face 72 of the core 100D placed into the volume 130 of the liquid disinfectant 34, and the liquid disinfectant 34 transporting along the capillary channels 140 due to the capillary effect (reflected by dashed lines).

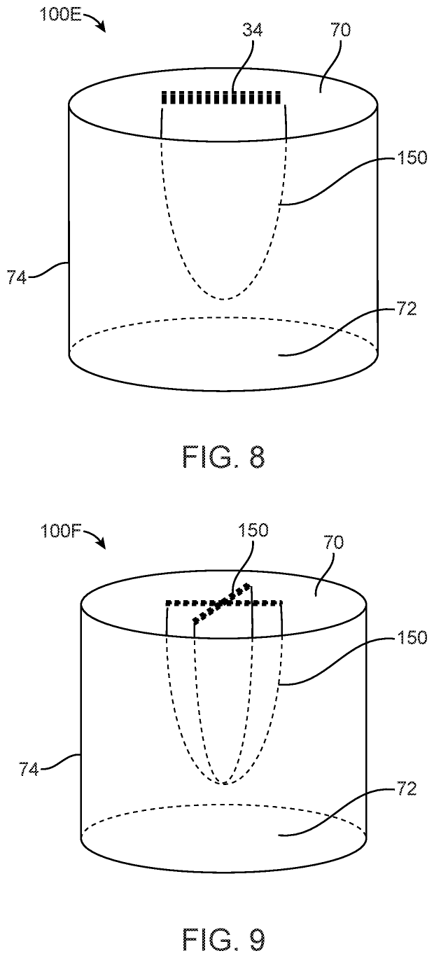

Another core 100E useful with the disinfecting devices of the present disclosure (e.g., in place of the core 42 of FIG. 1) is shown in FIG. 8. The core 100E is depicted as being transparent in the view of FIG. 8 to better illustrate the various features described below; it will be understood, however, that the material of the core 100E (e.g., a non-absorbent, closed-cell foam) need not be transparent in actual practice. The core 100E can be akin to the core 42 in terms of material and shape (e.g., defines the opposing exterior end faces 70, 72 and the exterior side face 74 as described above). In addition, the core 100E forms or defines one or more capillary channels 150. The capillary channel(s) 150 can be generally akin to the capillary channels described above, and are open to the first exterior end face 70. With the embodiment of FIG. 8, however, the capillary channel(s) 150 do not extend to, or are not open at, the second exterior end face 72 or the exterior side face 74. With these and related embodiments, the capillary channel(s) 150 can be described or referred to as a reservoir slit. As shown with dashed lines, the liquid disinfectant 34 is maintained and transported along the capillary channel 150 due to the capillary effect and is distributed at or along the first exterior end face 70. In other embodiments, two or more of the capillary channels 150 can be provided and that can intersect one another as shown, for example, with the core 100F of FIG. 9.

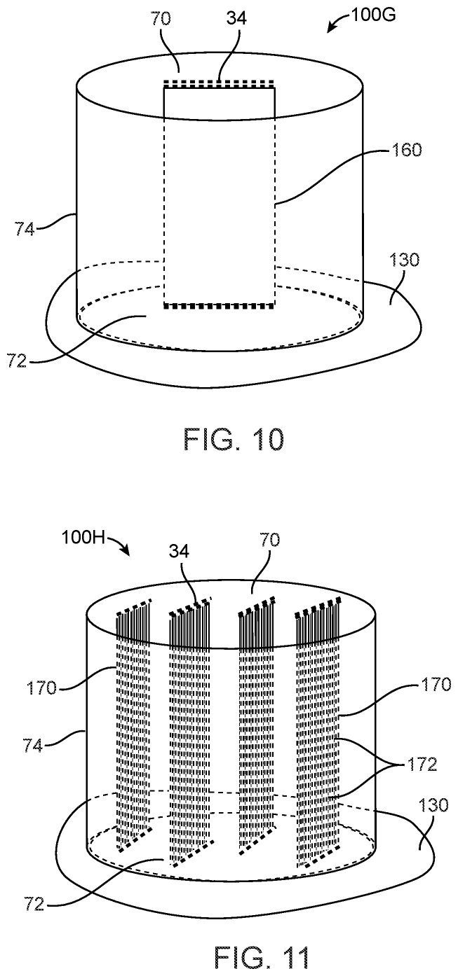

Another core 100G useful with the disinfecting devices of the present disclosure (e.g., in place of the core 42 of FIG. 1) is shown in FIG. 10. The core 100G is depicted as being transparent in the view of FIG. 10 to better illustrate the various features described below; it will be understood, however, that the material of the core 100G (e.g., a non-absorbent, closed-cell foam) need not be transparent in actual practice. The core 100G can be akin to the core 42 in terms of material and shape (e.g., defines the opposing exterior end faces 70, 72 and the exterior side face 74 as described above). In addition, the core 100G forms or defines one or more capillary channels 160. The capillary channel(s) 160 can be generally akin to the capillary channels 120 (FIG. 6) described above, and are open to the first and second exterior end faces 70, 72. With the embodiment of FIG. 10, however, the capillary channels 160 are not open to the exterior side face 74. With these and related embodiments, the capillary channel(s) 160 can be described or referred to as an interior capillary channel. While the capillary channel 160 is shown as being centrally disposed relative to a diameter of the core 100G, any other arrangement is equally acceptable. As a point of reference, FIG. 10 also depicts the capillary action as described above, with the second exterior end face 72 of the core 100G placed into the volume 130 of the liquid disinfectant 34, and the liquid disinfectant 34 transporting along the capillary channel 160 and distributed at or along the first exterior end face 70 due to the capillary effect (reflected by dashed lines).

Another core 100H useful with the disinfecting devices of the present disclosure (e.g., in place of the core 42 of FIG. 1) is shown in FIG. 11. The core 100H is depicted as being transparent in the view of FIG. 11 to better illustrate the various features described below; it will be understood, however, that the material of the core 100H (e.g., a non-absorbent, closed-cell foam) need not be transparent in actual practice. The core 100H can be akin to the core 42 in terms of material and shape (e.g., defines the opposing exterior end faces 70, 72 and the exterior side face 74 as described above). In addition, the core 100H forms or defines one or more capillary channel patterns 170, each comprised of a series of closely arranged capillary channels 172. The capillary channel pattern(s) 170 are open to the first and second exterior end faces 70, 72. With the embodiment of FIG. 11, the capillary channel patterns 170 are not open to the exterior side face 74. While four, equidistantly-spaced capillary channel patterns 170 are shown, any other number, and any other spacing format or pattern, is equally acceptable. As a point of reference, FIG. 11 also depicts the capillary action as described above, with the second exterior end face 72 of the core 100H placed into the volume 130 of the liquid disinfectant 34, and the liquid disinfectant 34 transporting along the capillary channel patterns 170 and distributed at or along the first exterior end face 70 due to the capillary effect (reflected by dashed lines).

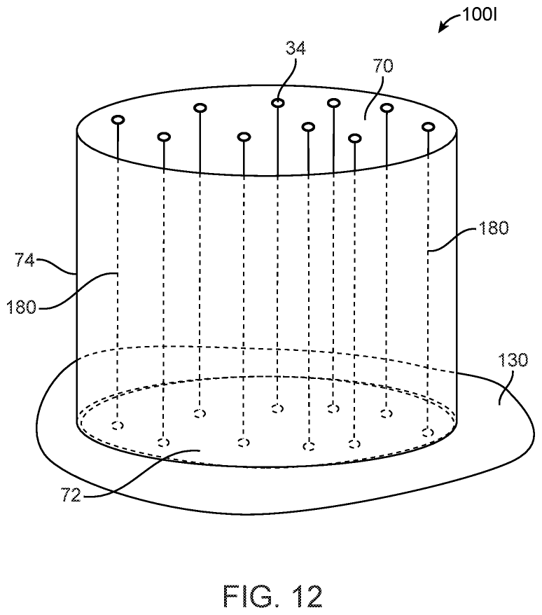

Another core 1001 useful with the disinfecting devices of the present disclosure (e.g., in place of the core 42 of FIG. 1) is shown in FIG. 12. The core 1001 is depicted as being transparent in the view of FIG. 12 to better illustrate the various features described below; it will be understood, however, that the material of the core 1001 (e.g., a non-absorbent, closed-cell foam) need not be transparent in actual practice. The core 1001 can be akin to the core 42 in terms of material and shape (e.g., defines the opposing exterior end faces 70, 72 and the exterior side face 74 as described above). In addition, the core 1001 forms or defines one or more capillary channels 180. The capillary channel(s) 180 can be generally akin to the capillary channels described above, and are open to the first and second exterior end faces 70, 72. As compared to some other embodiments, the capillary channels 180 can have a reduced size or cross-sectional shape, and in some embodiments are similar to a pin hole. With the embodiment of FIG. 12, the capillary channels 180 are not open to the exterior side face 74. While ten, randomly distributed capillary channels 180 are shown, any other number, and any other spacing format or pattern, is equally acceptable. As a point of reference, FIG. 12 also depicts the capillary action as described above, with the second exterior end face 72 of the core 1001 placed into the volume 130 of the liquid disinfectant 34, and the liquid disinfectant 34 transporting along the capillary channels 180 and distributed at or along the first exterior end face 70 due to the capillary effect (referenced generally in FIG. 12).

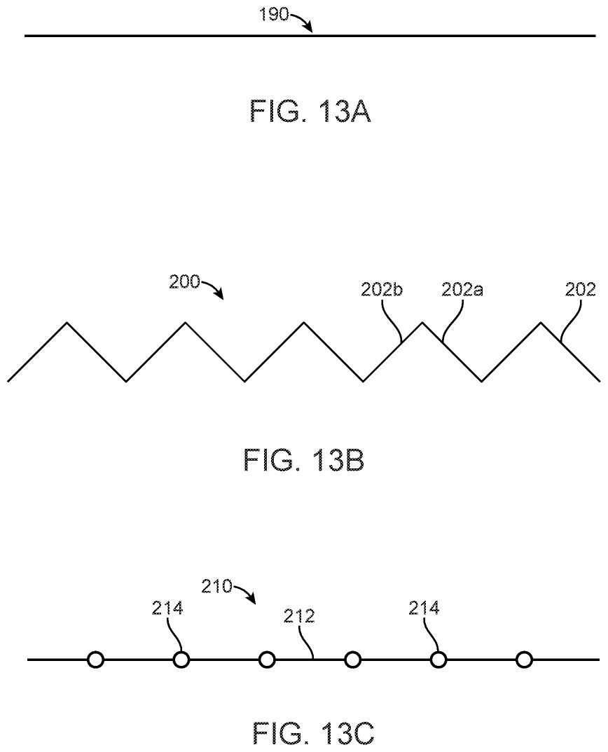

Returning to FIG. 1, the capillary channels of the present disclosure and useful with the core 42 (e.g., any of the capillary channel formats shown and described with respect to FIGS. 3A-12) can assume various other forms. Further, in some embodiments, a combination of differently sized and/or formatted capillary channels can be provided (e.g., the core 42 can include or incorporate a combination of two or more of the capillary channel formats described above). With embodiments in which the capillary channel has a slit-like format, a transverse shape of the capillary channel can assume various forms. For example, a representative shape of a capillary channel 190 as formed in a core of the present disclosure and as viewed from an end of the core (e.g., the first or second exterior end face 70, 72) is shown in FIG. 13A. The slit-like capillary channel 190 can be substantially straight or linear (e.g., within 10 percent of a truly linear shape) in extension across the core face. Capillary action patency can be optimize via selection of a precise length and/or width of the capillary channel 190.

A representative shape of another capillary channel 200 as formed in a core of the present disclosure and as viewed from an end of the core (e.g., the first or second exterior end face 70, 72) is shown in FIG. 13B. The slit-like capillary channel 200 can have the non-linear or zig-zag pattern like shape in extension across the core face as shown, effectively forming or defining a series of interconnected segments 202 that extend from one another in an angular fashion (e.g., first and second, immediately adjacent segment 202a, 202b are interconnected and arranged relative to one another to define an included angle of less than 180 degrees). Capillary action patency can be optimize via selection of a precise length and/or width of the segments 202.

A representative shape of another capillary channel 210 as formed in a core of the present disclosure and as viewed from an end of the core (e.g., the first or second exterior end face 70, 72) is shown in FIG. 13C. The capillary channel 210 can have a slit-like portion 212 (e.g., linear or non-linear) in extension across the core face as shown, along with one or more enlarged regions 214 intersecting the slit-like portion 212. In some embodiments, the capillary channel 210 of FIG. 13C can be generated by the capillary channel pattern 170 shown in FIG. 11.

Returning to FIG. 1, the housing 44 can assume various forms appropriate to facilitate user handling, and defines a cavity 220 sized and shaped to receive the element 40. With these and related embodiments, the housing 44 provides a barrier to direct contact with the element 40 when the disinfecting device 20 is in use, and serves as a tool or handling surface for mechanically manipulating the disinfecting device 20. In some embodiments, the housing 44 can be formed (e.g., molded) from a plastic material (e.g., a thermoformed plastic structure) and can be more rigid than a structure of the element 40. In some embodiments, the housing 44 is molded from a medical grade polypropylene or medical grade cyclic olefin copolymer (COC) material. In other embodiments, the housing 44 is molded from a medical grade high density polyethylene (HDPE) material. Further, the housing 44 can include or provide optional features that promote handling by a user's fingers or hands, such as grips or protrusions 222 formed on or by an exterior surface of the housing 44. Regardless, the cavity 220 is open at a rim 224 of the housing 44. The rim 224 can assume various forms, and is generally configured to (e.g., sized and shaped) to promote attachment of the lid 32, the rim 224 can form or define a relatively flat receiving surface. Other configurations or formats are also acceptable.

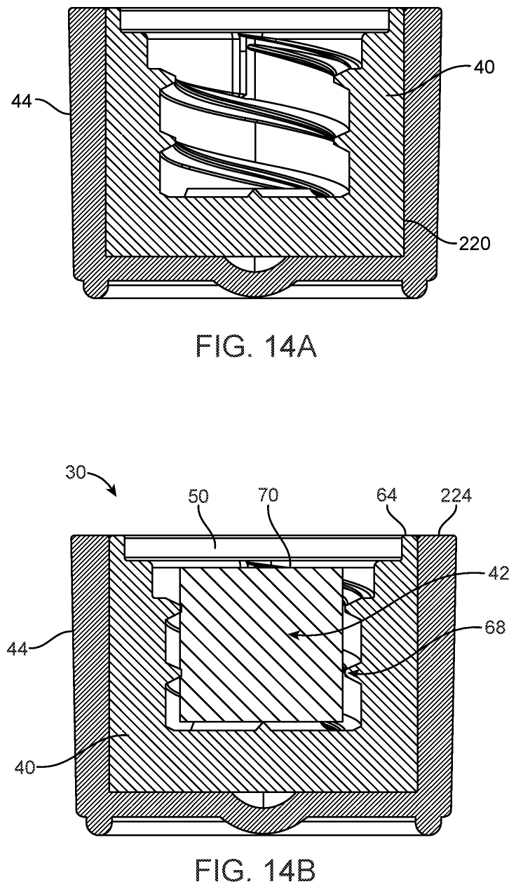

Construction of the interface assembly 30 is shown in the cross-sectional views of FIGS. 14A and 14B. In FIG. 14A, the element 40 has been placed or loaded into the cavity 220 (referenced generally) of the housing 44. In FIG. 14B, the core 42 has been placed or loaded into the chamber 50 of the element 40 (for ease of illustration, the capillary channel(s) of the core 42 are omitted from the view of FIG. 14B). As reflected by FIG. 14B, in some embodiments, a footprint (e.g., height and diameter) of the element 40 approximates a size of the cavity 220 such that upon final assembly, the leading end 64 of the element 40 is generally aligned with the rim 224 of the housing 44. The core 42, in turn, can have a width (e.g., diameter) approximating a diameter defined by the raised structures 68 (e.g., threads) formed by the element 40. In some embodiments, a height of the core 42 is less than a height of the cavity 220 such that upon final assembly, the first end face 70 of the core 42 is spaced away from (e.g., below relative to the orientation of FIG. 14B) the leading end 64 of the element 40. This optional spacing or gap facilitates insertion of a medical end-site (not shown) into the chamber 50 (via the leading end 64) prior to contacting the core 42.

Returning to FIG. 1, the lid 32 is sized and shaped in accordance with the housing 44, and in particular the rim 224, for assembly thereto. In some embodiments, the lid 32 can be a thin film or foil-type body, formed of a material conducive to sealing to the rim 224 of the housing 44, that can provide a sealed (e.g., hermetically sealed), sterile barrier to the cavity 220, and thus a sealed, sterile barrier to the chamber 50 of the element 42 (that is otherwise disposed within the cavity 220). For example, the lid 32 can be a film or foil that is sealed or otherwise attached to the rim 224 by conventional heat sealing techniques. In some embodiments, the lid 32 can form or provide one or more features that facilitate removal of the lid 32 from the housing 44 by a user. For example, the lid 32 can form or provide a lid body 230 and a pull tab 232. The lid body 230 is sized and shaped in accordance with a size and shape of the rim 224. The pull tab 232 extends from the lid body 230 and provides a convenient surface for grasping by a user when attempting to remove the lid 32 from the housing 44.

Final assembly of the disinfecting device 20 is shown in FIG. 15. A volume of the liquid disinfectant 34 (illustrated generally in FIG. 15) has been dispensed into the chamber 50 of the element 40 (e.g., prior to insertion of the core 42), and the lid 32 secured to the rim 224 of the housing 44. In this regard, the core 42 is in contact with the liquid disinfectant 34, with the capillary channel(s) (hidden in FIG. 15) of the core 42 transporting and distributing the liquid disinfectant 34 along at least a portion of the core 42, for example at or along the first end face 70, due to the capillary effect as described above. During use, the lid 32 is removed, and the interface assembly 30 manipulated (e.g., by user grasping the housing 44 with his/her fingers) to direct a medical end-site (not shown) into the chamber 50 of the element 40. As surfaces of the medical end-site are brought into contact with the liquid disinfectant 34 (e.g., by contacting the first end face 70 at which the liquid disinfectant has been transported and distributed), the liquid disinfectant 34 is transferred onto, and disinfects (either immediately or after drying) the so-contacted surfaces. The interface assembly 40 can be further manipulated relative to the medical end-site (and/or vice-versa) to scrub, wipe, or otherwise clean contacted surfaces. For example, where the medical end-site has or presents a threaded or similar surface, the interface assembly 30 can be rotated relative to the medical end-site (and/or vice-versa), bringing the raised structures 68 of the element 40 into intimate contact with the threaded surfaces and the rotating (or screwing) motion effecting a scrubbing-type action. Further, with advancement of the interface assembly 30 onto the medical end-site and subsequent motion of the interface assembly 30, a material of the core 42 otherwise in direct contact with the medical end-site (e.g., at the first end face 70) effects a scrubbing-type action on the so-contacted surface(s) of the medical end-site.

With other embodiments of the present disclosure, one or more capillary channels can be provided with, or formed by, the element 40 to effect transport and distribution of the liquid disinfectant 34 from the arrangement of FIG. 15. With these and related embodiments, the core 42 may or may not form capillary channel(s).

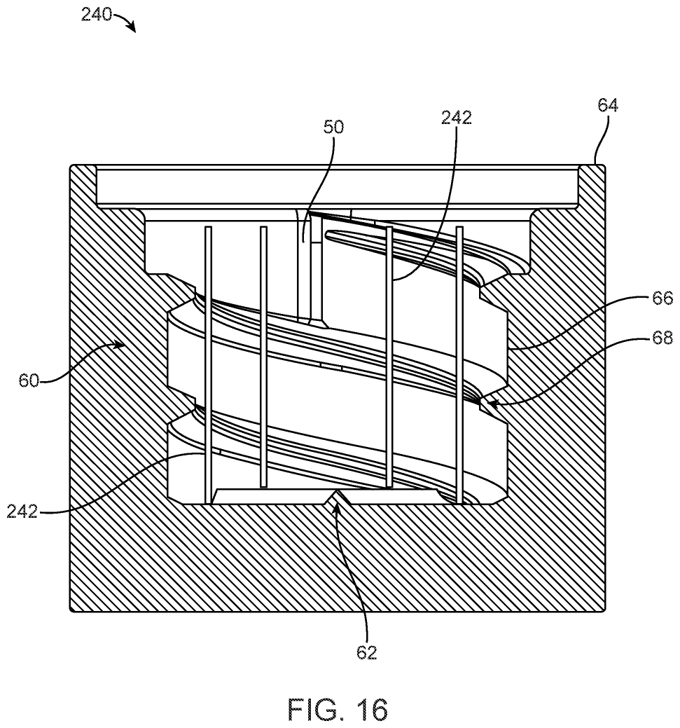

With this in mind, another embodiment of an element 240 useful with the interface assemblies and disinfecting devices of the present disclosure is shown in FIG. 16. The element 240 can be highly akin to the element 40 (FIG. 1) described above. For example, the element 240 can be a non-absorbent elastomeric material body (e.g., thermoplastic vulcanized rubber), and forms or defines the chamber 50 and the raised structures 68 (e.g., threads) along the inner surface 66 thereof. In addition, the element 240 forms or provides one or more capillary channels 242 along the inner surface 66. The capillary channel(s) 242 can have any of the formats described elsewhere, and is/are a narrow void formed into the inner surface 66 for absorbing, transporting, and retaining a disinfectant liquid (not shown) along the solid inner surface 66 via capillary action (i.e., the liquid wicks due to fine, narrow channels facilitating molecular surface tension and the ensuing capillary action sufficient to retained and transport (migrate) the liquid along a length of the inner surface 66 on the element 240). The capillary channel(s) 242 distributes the liquid along the inward facing inner surface 66 of the non-absorbent elastomeric, solid material element 240 that can otherwise have compression and rebound properties. For example, when the element 240 is engaged with a medical end-site, the capillary channel 242 initiates transportation of the antimicrobial liquid during engagement and application of the element 240 with the medical end-site such that contact by a twisting or pushing the medical end-site into the element 240 initiates “capillary action” and transportation of the antimicrobial.

The capillary channel(s) 242 is, in some embodiments, open to chamber 50. The capillary channel(s) 242 can be formed into and along the solid material inner surface 66 as a cut, a groove, a sipe, split, a slit, a scoring, an indent, a notch, a channel, incision, fissure, etc. Various techniques can be employed to generate the capillary channel(s) 242 for example, but not limited to, cutting, molding, laser, razor, plasma etching or plasma treatment, die-cut, waterjet, scoring, tearing, lancing, incising, etc. As shown, one or more of the capillary channels 242 can pass through (and this “into”) one or more of the raised structures 68.

While the capillary channels 242 are shown as being relatively linear or uniform, other configurations are also acceptable. The opposing long edges of one or more of the capillary channels 242 can comprise equal margins or irregular margins (i.e., the width or distance between the margins are the same or different widths along a length thereof). In some embodiments, one or more of the capillary channels 242 are formed such that when the element 240 is at rest or in a normal state (e.g., not inserted over a medical end-side), the corresponding long edges or margins touch one another and partially close or seal the capillary channel 242 and splay open upon contact by a medical end-site. With these and related embodiments, during use the capillary channel 242 splays-open upon contact with a medical end-site to effect dynamic surface cleaning and dynamic scrubbing of the medical end-site surface (as compared to a solid surface that omits the capillary channel 242). In some embodiments, the capillary channel 242 is configured to self-seal prior to engagement with a medical end-site and opens during application and/or contact (such as by twisting or pushing the medical end-site into the element 240) and once physically attached to the medical end-site the capillary channel 242 closes to re-seal while the element 240 remains secured to the medical end-site. In other embodiments, the capillary channel 242 is configured to self-seal prior to engagement with a medical end-site and opens during application and/or contact (such as by twisting or pushing the medical end-site into the element 240) and once physically attached to the medical end-site the capillary channel 242 remains open while the element 240 remains secured to the medical end-site. In other embodiments, the capillary channel 242 is configured to self-seal prior to engagement with a medical end-site and opens during application and/or contact (such as by twisting or pushing the medical end-site into the element 240) and once physically engaged to the medical end-site the capillary channel 242 initiates capillary action while the element 240 remains secured to the medical end-site.

In other embodiments, the capillary channel 242, as formed in the resilient material of the element 240, is self-sealing (e.g., the elongated open-surface capillary channel 242 with the element 240 at rest is closed, or partially closed/slightly open, to be sealed at or along a margin of the capillary channel 242). When the capillary channel 242 is engaged with a medical end-site it opens and the material of the element 240 is sufficiently resilient for the capillary channel 242 to rebound and return to a self-sealed state and/or at a near its original closed position with the medical end-site still engaged. Alternatively or in addition, the capillary channel 242 can be figured such that when the medical end-site has been disengaged with the inner surface 66, the capillary channel 242 returns to a self-sealed state and/or at a near its original state.

The capillary channel(s) 242 can be formed at a variety of different depths. For example, in some embodiments, the capillary channel 242 transects a contour of the element 240 (e.g., penetrating the raised structure 68 but not cutting deep enough to penetrate the side wall 60). In other embodiments, the capillary channel 242 transects a structural contour (e.g., transecting the raised structure 68) in combination with the side wall 60. With these and related embodiments, the depth of the capillary channel 242 is greater as it passes through the structural contour and a lesser depth as it passes through the adjoining side wall 60.

In some embodiments, the capillary channel(s) 242 is a “continuous” channel in that it is open at both ends. For example, the capillary channel 242 along the vertical side wall 60 of the element 240 to the base 62 at which an end of the capillary channel 242 is open, and the opposite end of the capillary channel 242 is also open at the leading end 64 of the element 240. In other embodiments, the capillary channel 242 is a “terminal” channel that has only one open end as the channel extends across a length of the inner surface 66, and is other closed/terminates at the opposite end. For example, the capillary channel 242 can be formed along inside of the side wall 60 with one end that interfaces/terminates at the base 62 and the opposing end is open at or near the leading end 64 of the element 240.

While the capillary channel(s) 242 is shown as traversing a near entirety of a length of the chamber 50, other configurations are also acceptable. In other embodiments, one or more of the capillary channels 242 can be formed to traverse a limited or specific region along the chamber 50. For example, the capillary channel 242 can extend from or along the base 62 only; or the upper inner side wall 60; or the exterior side wall 60; or only the lower to mid-area on the inner side wall 60; or a combination of a raised base 62 and the lower inner side wall 60 or a combination of the leading end 64 and the upper inner side wall 60 excluding the base of the element 60; or the entirety of the chamber 50 where the entire inner surface 66, consisting of form-fitting contours, but excluding an exterior surface of the element 240; or the entirety of the exterior surface of the element 240; or only specific structural aspect on the exterior of the element 240.

With the above in mind, some examples of a regional surface having a specific open-surface capillary channel 242 is the raised base 62. In some embodiments the raised base 62 comprises one or more elongated open-surface capillary channels 242 that crisscross the surface of the structure that comprises the raised base 62 and sufficiently retains the liquid antimicrobial via capillary action or in a combination with surface tension and capillary action. The raised base 62, with the capillary channel(s) 242, can further function as a “dynamic scrubbing surface” as the capillary channel(s) 242 interface with a medical end-site surface such as a septum. With these and related embodiments, the crisscross raised base 62 dynamically moves to dynamically conform and uniformly correspond to the features on the medical end-site septum. The dynamic scrubbing occurs when the element 240 interfaces with the medical end-site and is rotated, twisted, screwed back and forth or compressed all while simultaneously applying the antimicrobial liquid.

Other examples of a regional surface having an open-surface capillary channel 242 is at or along the side wall 60. With these and related embodiments, the so-provided capillary channel 242 is capable of both delivery of the antimicrobial liquid and a dynamic scrubbing as well as removal and cleaning of debris from the surfaces of the medical end-site.

While the capillary channels 242 are shown as being linear and discrete from one another, other configurations are also acceptable. The capillary channels 242 can be linear, crisscross, circular, circumferential, crosshatch or bisecting one another in some embodiments. In some embodiments, the capillary channel(s) 242 follows a contour-line of the raised structure(s) 68 such that the capillary channel 242 traces along the length of the raised structure 68 (as opposed to transecting the raised structure 68 perpendicularly) to divide the raised structure 68 along the length (e.g., dividing along the length of a formed thread on the inner surface 66 of the element 240 and around circumferentially the thread path) and whereby the capillary channel 240 splits along the “spine” of the raised structure 68 (e.g., thread) such that the raised structure 68 is divided or halved along the length. In other embodiments, the capillary channel 242 transects the raised structure(s) 68 as a contour and/or into segments (e.g., a circumferential flange raised structure is divided/transected and forms segments by multiple capillary channels 242 transecting the flange around a circumference on the side wall 60 of the element 240). In other embodiments, a thread formed along the chamber 50 can be transected by multiple capillary channels 242 that bisect and segment the structural thread contour to form angular, stepped segments of the thread shape.

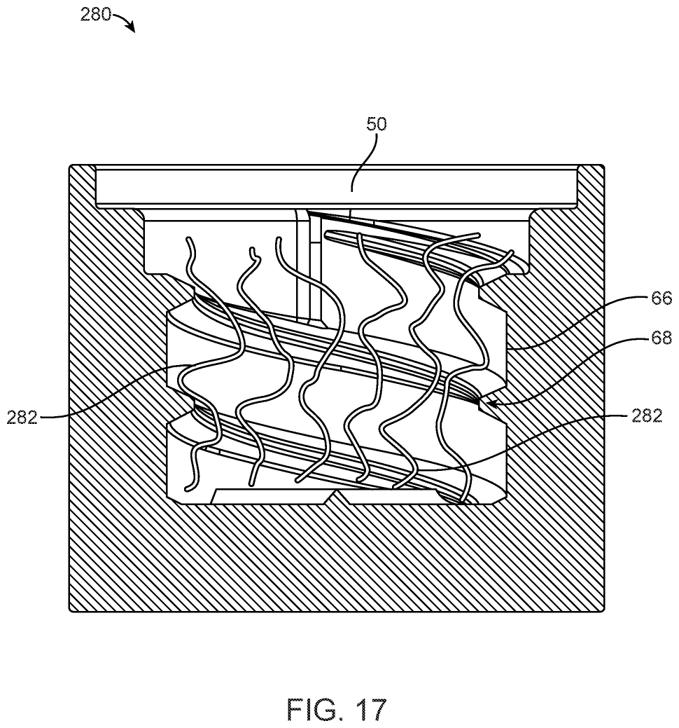

In yet other embodiments, the capillary channels 242 can have a less uniform shape (as compared to the shape reflected by FIG. 16) and/or two or more of the capillary channels 242 may intersect one another. For example, another embodiment of an element 280 useful with the interface assemblies and disinfecting devices of the present disclosure is shown in FIG. 17. The element 280 can be highly akin to the element 40 (FIG. 1) described above. For example, the element 280 can be a non-absorbent elastomeric material body (e.g., thermoplastic vulcanized rubber), and forms or defines the chamber 50 and the raised structures 68 (e.g., threads) along the inner surface 66 thereof. In addition, the element 280 forms or provides one or more capillary channels 282 along the inner surface 66. The capillary channels 282 can have the non-uniform shape in extension along the element 280 as reflected by the view. Apart from the non-uniform shape, the capillary channels 282 can have any of the configurations described above with respect to the capillary channels 242 (FIG. 16).

Returning to FIG. 1, with other embodiments of the present disclosure, one or more capillary channels can be provided with, or formed by, the housing 44 to effect transport and distribution of the liquid disinfectant. With these and related embodiments, the element 40 and/or core 42 may or may not form capillary channel(s). Moreover, with embodiments in which the housing 44 forms or defines one or more capillary channels, the housing alone or the housing carrying the element 40 (and not including the core 42) can serve as the interface assembly of the corresponding disinfecting device. In yet other embodiments, the element 40 can be omitted, with the interface assembly of the corresponding disinfecting device comprising the core 42 maintained within the housing 44.

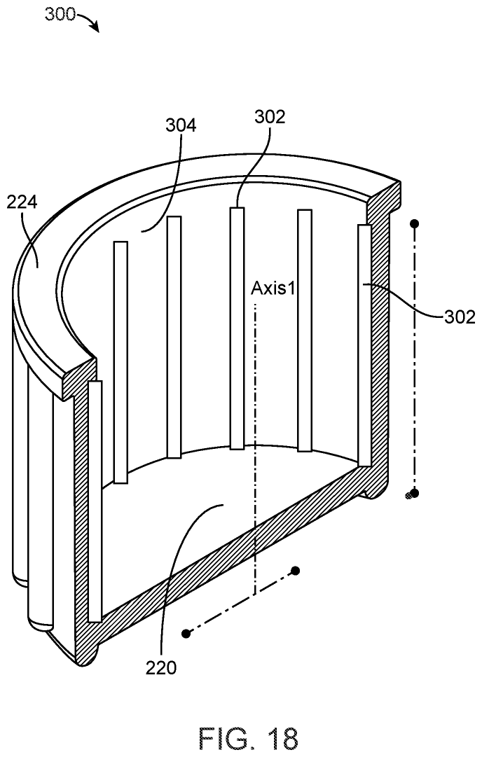

With this in mind, another embodiment of a housing 300 useful with the interface assemblies and disinfecting devices of the present disclosure is shown in FIG. 18. The housing 300 can be highly akin to the housing 44 (FIG. 1) described above. For example, the housing 300 can be a molded plastic material that forms or defines the cavity 220 and the rim 224. In addition, the housing 300 forms or provides one or more capillary channels 302 along an interior surface 304 (that otherwise extends from a floor 306 to the rim 224). The capillary channel(s) 302 can have any of the formats described elsewhere, and is/are a narrow void formed into the interior surface 304 for absorbing, transporting, and retaining a disinfectant liquid (not shown) along the solid interior surface 304 via capillary action (i.e., the liquid wicks due to fine, narrow channels facilitating molecular surface tension and the ensuing capillary action sufficient to retained and transport (migrate) the liquid along a length of the inner surface 304 on the housing 300). The capillary channel(s) 302 distributes the liquid along the inward facing interior surface 304. For example, when the housing 300 is engaged relative to a medical end-site, the capillary channel 302 initiates transportation of the antimicrobial liquid during engagement and application of the housing 300 with the medical end-site such that contact by a twisting or pushing the medical end-site into the housing 300 initiates “capillary action” and transportation of the antimicrobial.

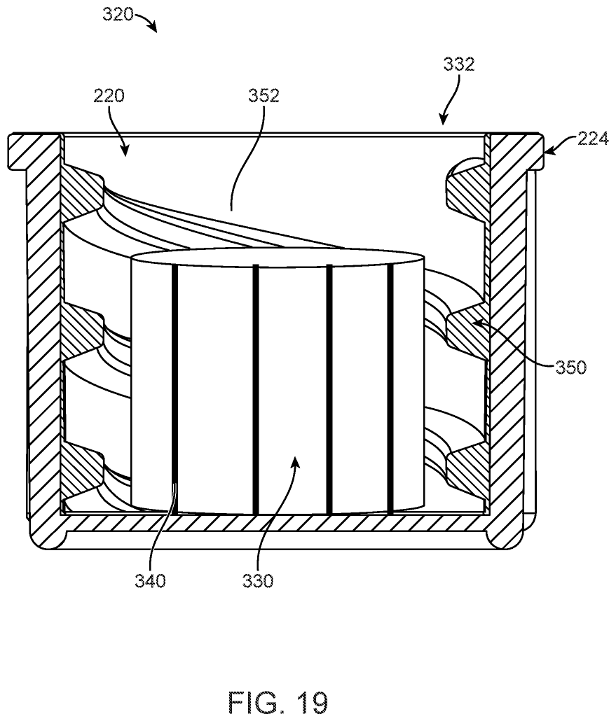

FIG. 19 is a side sectional view of another interface assembly 320 in accordance with principles of the present disclosure. The interface assembly 320 includes a core 330 and a housing 332. As compared to the interface assembly 30 (FIG. 1), the interface assembly 320 does not include an element (e.g., the element 40 of FIG. 1 is omitted). With this in mind, the core 330 can assume any of the formats described elsewhere. For example, the core 330 can be akin to the core 42 (FIG. 1) in terms of material and shape (e.g., a non-absorbent, closed-cell foam), and defines one or more capillary channels 340 that can have any of the formats described elsewhere. The housing 332 can be highly akin to the housing 44 (FIG. 1) described above. For example, the housing 332 can be a molded plastic material that forms or defines the cavity 220 and the rim 224. In addition, the housing 332 forms or provides one or more threads or similar raised structures 350 along an interior surface 352. The threads or raised structures 350 project into the cavity 220, and are configured to engage the threads, sides, and/or edges on the working end-site. In some embodiments, the raised structures 350 can include threads, ridges, flanges, steps, nubs, tines, bumps, etc., or combinations thereof. The interior surface 352 can optionally define a tapering diameter (either stepped change in diameter or a more uniformly tapering diameter). Regardless, the threads or raised structures 350 can facilitate the effective cleaning (and sterilization) of surfaces of the medical end-site by directly contacting and conforming to surfaces of interest.

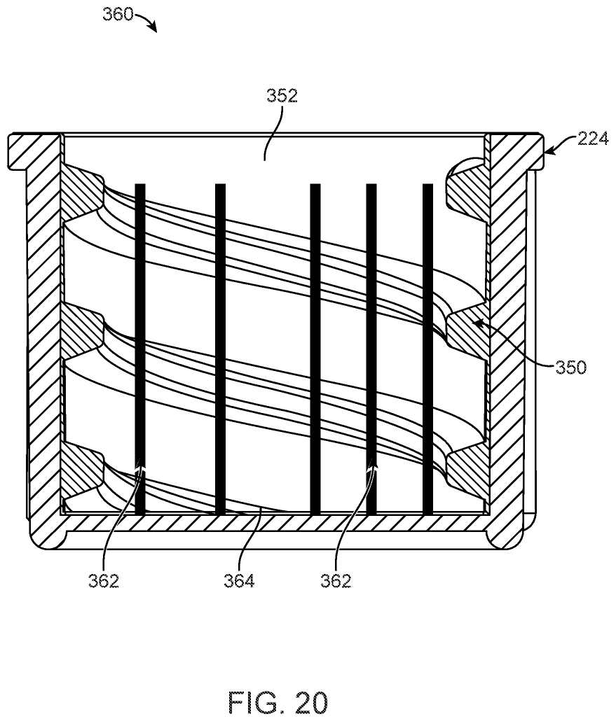

FIG. 20 is a side sectional view of another housing 360 useful with, or as, the interface assemblies of the present disclosure. The housing 360 can be highly akin to the housing 332 (FIG. 19) described above. For example, the housing 360 can be a molded plastic material that forms or defines the cavity 220, the rim 224, and the one or more threads or similar raised structures 350 along the interior surface 352 as described above. In addition, the housing 360 forms or provides one or more capillary channels 362 along the interior surface 352 (that otherwise extends from a floor 364 to or towards the rim 224). The capillary channel(s) 362 can have any of the formats described elsewhere, and is/are a narrow void formed into the interior surface 352 for absorbing, transporting, and retaining a disinfectant liquid (not shown) along the solid interior surface 352 via capillary action (i.e., the liquid wicks due to fine, narrow channels facilitating molecular surface tension and the ensuing capillary action sufficient to retained and transport (migrate) the liquid along a length of the inner surface 352 on the housing 360). The capillary channel(s) 362 distributes the liquid along the inward facing interior surface 352. For example, when the housing 360 is engaged relative to a medical end-site, the capillary channel 362 initiates transportation of the antimicrobial liquid during engagement and application of the housing 360 with the medical end-site such that contact by a twisting or pushing the medical end-site into the housing 360 initiates “capillary action” and transportation of the antimicrobial.

In some embodiments, an interface assembly of the present disclosure omits the element 40 (FIG. 1) and consists of the housing 360 maintaining a core (not shown, but in some embodiments akin to the core 42 (FIG. 1). With these and related embodiments, the core maintained by the housing 360 may or may not include capillary channels.

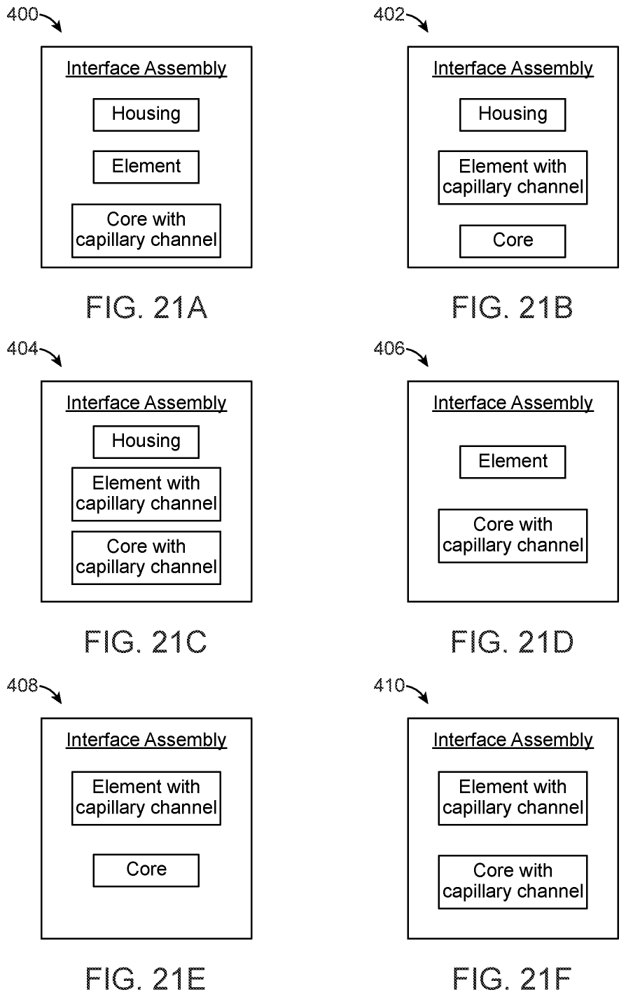

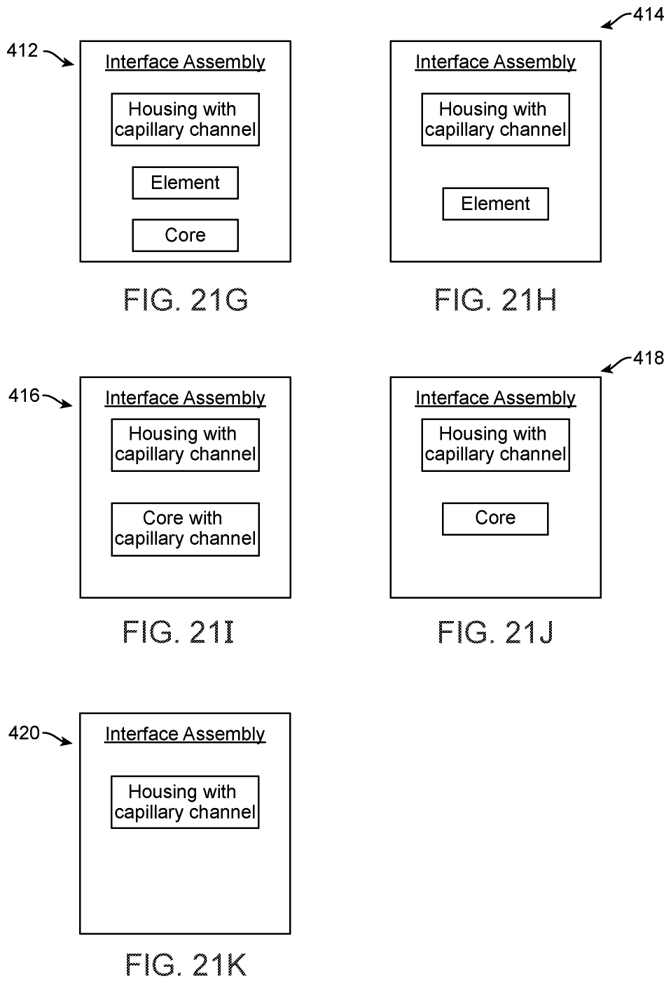

From the above explanations, the interface assemblies of the present disclosure, and thus the disinfecting devices of the present disclosure, can assume a variety of forms. As summarized in FIGS. 21A-21, interface assemblies of the present disclosure include: An interface assembly 400 (FIG. 21A) comprised of a housing, an element, and a core with at least one capillary channel; an interface assembly 402 (FIG. 21B) comprised of a housing, an element with at least one capillary channel, and a core; an interface assembly 404 (FIG. 21C) comprised of a housing, an element with at least one capillary channel, and a core with at least one capillary channel; an interface assembly 406 (FIG. 21D) comprised of an element and a core with at least one capillary channel (housing omitted); an interface assembly 408 (FIG. 21E) comprised of an element with at least one capillary channel and a core (housing omitted); an interface assembly 410 (FIG. 21F) comprised of an element with at least one capillary channel and a core with at least one capillary channel (housing omitted); an interface assembly 412 (FIG. 21G) comprised of a housing with at least one capillary channel, an element, and a core; an interface assembly 414 (FIG. 21H) comprised of a housing with at least one capillary channel and an element (core omitted); an interface assembly 416 (FIG. 21I) comprised of a housing with at least one capillary channel and a core with at least one capillary channel (element omitted); an interface assembly 418 (FIG. 21J) comprised of a housing with at least one capillary channel and a core (element omitted); and an interface assembly 420 (FIG. 21K) comprised of a housing with at least one capillary channel (core and element omitted).

Although the present disclosure has been described with reference to preferred embodiments, workers skilled in the art will recognize that changes can be made in form and detail without departing from the spirit and scope of the present disclosure.

What is claimed is:

1. A medical end-site disinfecting device comprising:
an interface device for interfacing with a medical end-site, the interface device including at least one of:
a housing,
an element,
a core;
wherein at least one component of the interface device defines at least one capillary channel; and
a volume of a liquid disinfectant retained along the capillary channel;
wherein the at least one capillary channel is configured to generate a capillary effect to transport the liquid disinfectant along the interface device.

2. The disinfecting device of claim 1, wherein the component of the interface device defining the at least one capillary channel is formed of a non-absorbent material.

3. The disinfecting device of claim 2, wherein the at least one capillary channel is formed on a surface of the non-absorbent material.

4. The disinfecting device of claim 1, wherein the interface device includes the core disposed within a chamber of the element.

5. The disinfecting device of claim 4, wherein the core is formed of a non-absorbent closed-cell foam, and the element is formed of a non-absorbent elastic material.

6. The disinfecting device of claim 5, wherein the core defines at least one capillary channel.

7. The disinfecting device of claim 5, wherein the element defines at least one capillary channel.

8. The disinfecting device of claim 5, wherein the core defines at least one capillary channel and the element defines at least one capillary channel.

9. The disinfecting device of claim 5, wherein the interface device further includes the housing, and further wherein the element is disposed within a cavity of the housing.

10. The disinfecting device of claim 5, wherein the core defines a plurality of capillary channels.

11. The disinfecting device of claim 5, wherein the at least one capillary channel is defined as one of a cut, slit, score, sipe, groove, indent, impression, notch, channel, incision, interstice, crevice, cranny, fissure, and depression into a thickness of the core.

12. The disinfecting device of claim 5, wherein the at least one capillary channel is formed in, and is open relative to, an exterior surface of the core.

13. The disinfecting device of claim 12, wherein the core has a cylindrical shape defining opposing first and second end faces and a side face, and further wherein the at least one capillary channel is open to at least one of the first end face, the second end face, and the side face.

14. The disinfecting device of claim 13, wherein the core defines a plurality of capillary channels, and further wherein each of the plurality of capillary channels are open to at least two of the first end face, the second end face, and the side face.

15. The disinfecting device of claim 1, wherein the interface device includes the element disposed within a cavity of the housing.

16. The disinfecting device of claim 15, wherein the element is formed of a non-absorbent elastic material, and the housing is formed of a non-absorbent plastic material.

17. The disinfecting device of claim 15, wherein the element defines at least one capillary channel.

18. The disinfecting device of claim 15, wherein the housing defines at least one capillary channel.

19. The disinfecting device of claim 1, wherein the interface device includes the core disposed within a cavity of the housing.

20. The disinfecting device of claim 1, wherein the interface assembly consists of the housing, and further wherein the housing defines the at least one capillary channel.

21. The disinfecting device of claim 1, further comprising a lid removably secured to the interface assembly.
